(19) United States
(12) Patent Application Publication
(10) Pub. No.: US 2026/0284303 A1
(43) Pub. Date: Sep. 24, 2026

(54) Syringe Pump and Systems and Methods of Using the Same

(71) Applicant: UAB RESEARCH FOUNDATION, Birmingham, AL (US)

(72) Inventors: Harrison Kim, Birmingham, AL (US); Martin Holland, Homewood, AL (US)

(21) Appl. No.: 18/996,121

(22) PCT Filed: Jul. 19, 2023
(86) PCT No.: PCT/US2023/028143

§ 371 Date: Jan. 17, 2025

Related U.S. Application Data
(60) Provisional application No. 63/390,524, filed on Jul. 19, 2022.

(51) Int. Cl. A61M 5/145; A61M 5/14; A61M 5/168

(52) U.S. Cl. CPC A61M 5/145; A61M 5/1408; A61M 5/168; A61M 2005/14506; A61M 2205/057

(57) ABSTRACT

A syringe pump made of non-metallic material. The syringe pump having a spring, a rack, an engagement structure, and a syringe holder. The rack connects to the spring. The rack moves between a first position and a second position as the spring compresses and expands. The engagement structure is movably coupled to the rack. The engagement structure is configured to control a speed at which the rack moves between the first position and the second position. The syringe holder is configured to receive a syringe. The rack is configured to engage with a plunger of the syringe. The rack is configured to apply a force to the plunger as the rack moves from the first position to the second position to drive the plunger i

### CROSS REFERENCE TO RELATED APPLICATIONS

This application claims the benefit of the filing date of U.S. Provisional Application No. 63/390,524, which was filed on Jul. 19, 2022. The content of this earlier filed application is hereby incorporated by reference herein in its entirety.

### FIELD

Disclosed herein is a syringe pump that may be used within the same room as an imaging device, or further, may be positioned within a bore of the imaging device, while the imaging device is in use.

### BACKGROUND

Imaging devices, such as a Magnetic Resonance Imaging (MRI) scanner, often require a fluid contrast agent to be delivered to the subject during the imaging procedure for enhanced contrast in the resulting images. Further, some images require the fluid contrast agent to be delivered to multiple subjects at the same time. For example, the fluid contrast agent may need to be delivered to multiple animals within the bore of the MRI scanner in a preclinical imaging study. Further, the contrast agent may need to be delivered to a subject/patient and to an imaging phantom that may be used to normalize the obtained data at the same time.

The contrast agent is typically delivered with an electrically powered syringe pump. MRI-compatible syringe pumps and non-MRI compatible syringe pumps are available, but these syringe pumps have several disadvantages. Although an MRI-compatible syringe pump may be used inside the MRI room, the conventional MRI-compatible syringe pumps are expensive (costing approximately $100,000), bulky, and only inject the contrast agent into one subject at a time. While a non-MRI compatible syringe pump may inject contrast agents into multiple subjects at a controlled injection rate, the non-MRI compatible syringe pumps must be placed outside the MRI room because these syringe pumps include metals that interfere with the magnetic field of the MRI machine. Non-MRI compatible syringe pumps typically require 5-10 m long tubes to connect the syringes outside the MRI room to the subject(s) inside the MRI room. Long tubes require additional contrast agent to fill the inner space of the tubes. For example, if only 0.2 ml is necessary for an injection, 20 ml of contrast agent may be required to fill up a 10 m long tube. Further, conventional syringe pumps are expensive and must be programmed, requiring additional training for technologists/technicians.

Thus, there is a need for a syringe pump that addresses one or more of the deficiencies of existing syringe pumps. For example, there is a need for a syringe pump that is MRI-compatible, may inject contrast agents into multiple subjects at a controlled injection rate, may not require programming, is lightweight, and is less expensive.

### SUMMARY

Described herein, in various aspects, is a syringe pump. The syringe pump may be an MRI-compatible syringe pump that consists of non-metallic material. The syringe pump may be made of plastics. The syringe pump may have a spring, a rack, an engagement structure, and a syringe holder. The spring may be configured to move between a compressed position and an uncompressed position. The rack may connect to the spring. The rack may be configured to move between a first position and a second position via the spring. The first position and the second position of the rack may correspond to the compressed position and the uncompressed position of the spring, respectively. The engagement structure may be movably coupled to the rack. The engagement structure may be configured to control a speed at which the rack moves between the first position and the second position. The syringe holder may be configured to receive a syringe. The rack may be configured to engage with a plunger of the syringe. The rack may be configured to provide force to the plunger as the rack moves from the first position to the second position to drive the plunger into the syringe. Systems and methods of using the disclosed syringe pump are also described.

### DESCRIPTION OF THE DRAWINGS

FIG. 1 is a photograph of an exemplary syringe pump and syringe as disclosed herein.

FIG. 1A is a photograph of an exemplary syringe pump and syringes as disclosed herein.

FIG. 1B is an exploded view of the exemplary syringe pump of FIG. 1A.

FIG. 2 is a photograph of an exemplary rack of the exemplary syringe pump of FIG. 1.

FIG. 3 is a photograph of an exemplary syringe holder of the exemplary syringe pump of FIG. 1.

FIGS. 4A-4B are photographs of an exemplary installation of a syringe in the exemplary syringe holder of FIG. 1.

FIG. 5 is a photograph of an exemplary spring of the exemplary syringe pump of FIG. 1.

FIGS. 6A-6B are photographs of an exemplary lock of the exemplary syringe pump of FIG. 1. FIG. 6A shows the exemplary syringe pump in an unlocked and unloaded position. FIG. 6B shows the exemplary syringe pump in a locked and loaded position.

FIG. 6C is a photograph of an exemplary lock of the exemplary syringe pump of FIG. 1A.

FIGS. 7A-7B are photographs of an exemplary controller of the exemplary syringe pump of FIG. 1. FIG. 7A shows the controller prior to being activated. FIG. 7B shows the controller after being activated.

FIG. 7C-7D are section views of an exemplary controller of the exemplary syringe pump of FIG. 1A. FIG. 7C shows the controller prior to being activated. FIG. 7D shows the controller after being activated.

FIG. 8 is a photograph of an exemplary engagement structure of the exemplary syringe pump of FIG. 1.

FIGS. 8A and 8B are photographs of an exemplary engagement structure of the exemplary syringe pump of FIG. 1A. FIG. 8A shows the engagement structure without a speed controller. FIG. 8B shows an exemplary speed controller.

FIGS. 9A-9B are photographs of an exemplary syringe pump with a syringe installed. FIG. 9A shows the syringe pump and syringe in a loaded position prior to the syringe pump being activated or used. FIG. 9B shows the syringe pump and syringe in an unloaded position after the syringe pump is used to dispense a contrast agent from within the barrel of the syringe.

FIGS. 10A-10C are photographs of an exemplary modification to an exemplary syringe pump wherein the syringe pump includes multiple syringes, multiple springs, and an enlarged rack. FIG. 10A is a photograph of the syringe pump, FIG. 10B is a photograph showing exemplary springs for use in the syringe pump, and FIG. 10C is a photograph of three syringes loaded into the syringe pump.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains, having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a”, “an”, and “the” can optionally include plural referents unless the context clearly dictates otherwise. For example, use of the term “a syringe” may refer to one syringe or a plurality of such syringes unless the context indicates otherwise.

All technical and scientific terms used herein have the same meaning as commonly understood to one of ordinary skill in the art to which this invention belongs unless clearly indicated otherwise.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

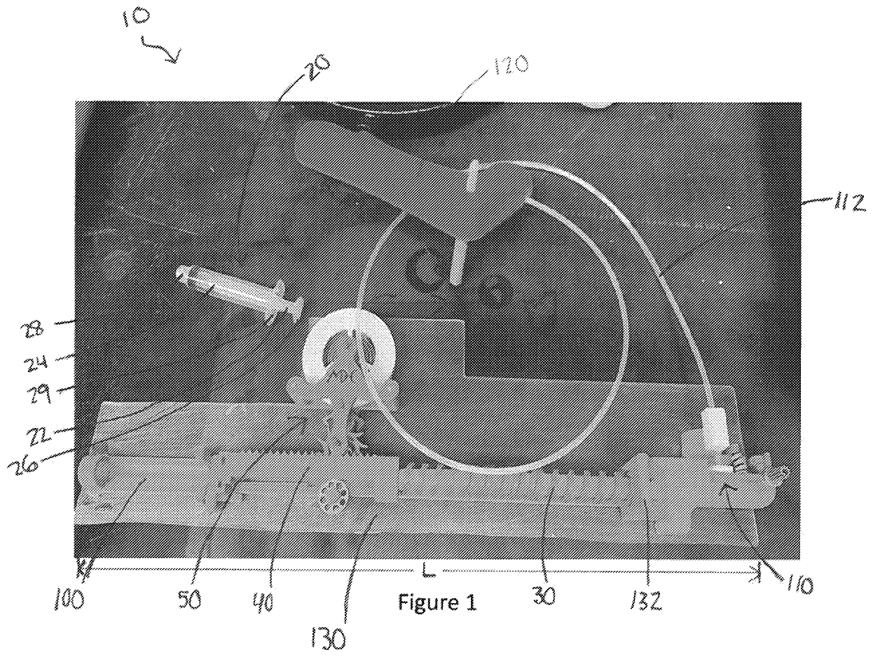

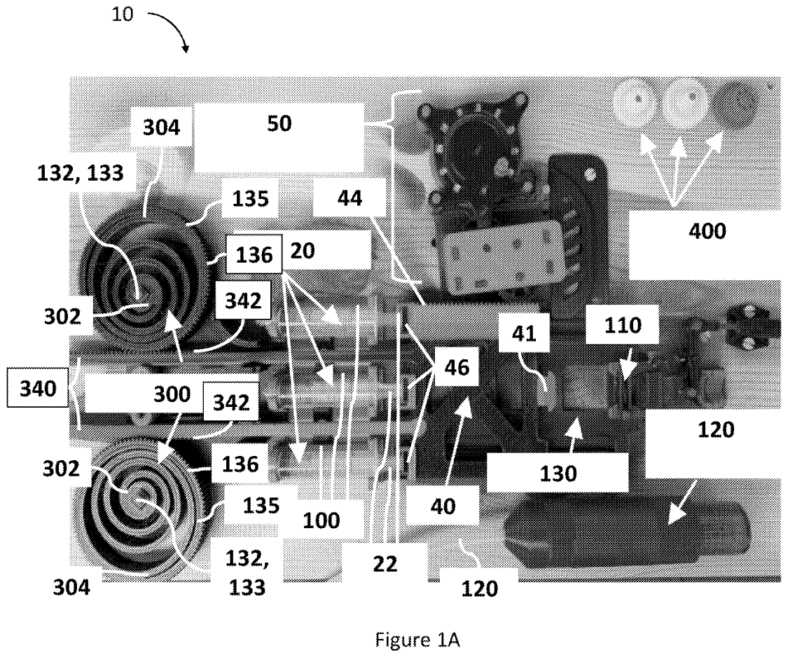

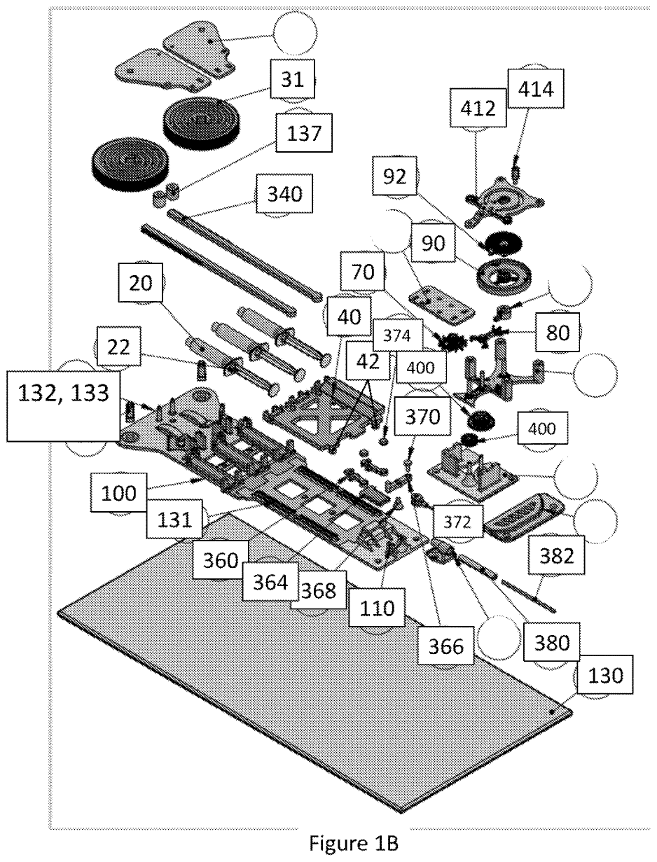

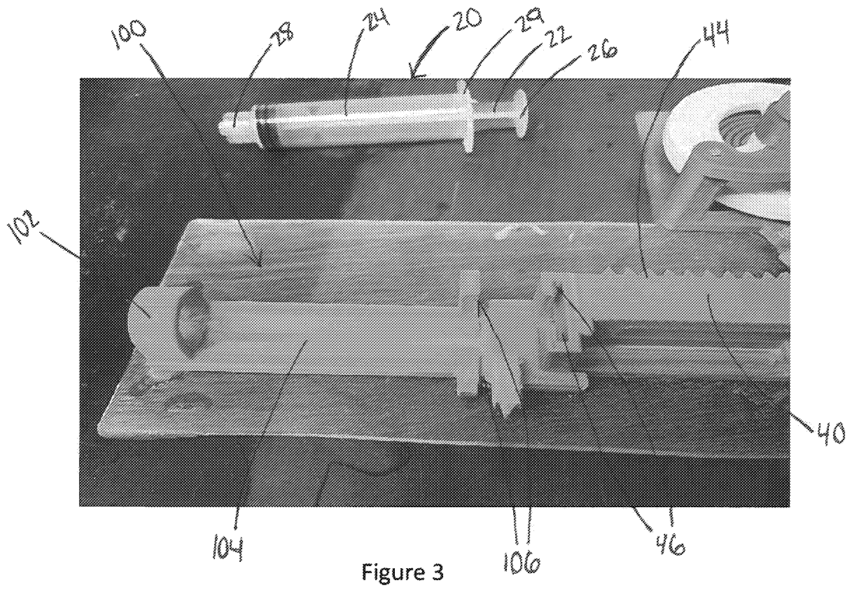

Disclosed herein with reference to FIGS. 1-10C is a syringe pump 10. The syringe pump 10 may comprise, be made of, or consist of non-metallic and/or non-magnetic material. For example, the syringe pump 10 may be made of plastics. The material and structure of the syringe pump 10 allow the device to be lightweight, portable, and less expensive to manufacture than conventional syringe pumps. The material and structure also allow the syringe pump 10 to be compatible with imaging devices. In exemplary uses, the syringe pump 10 may be configured for an MRI scanner. However, it is contemplated that the syringe pump 10 may be used with other imaging modalities, including, for example and without limitation, fluoroscopy, computed tomography (CT), or dynamic positron emission tomography (PET). The material and structure may allow the syringe pump 10 to be compatible with imaging devices, for example the syringe pump 10 may be MRI-compatible, because the material of the syringe pump 10 does not interfere with or affect the magnetic field of the machine. The syringe pump 10 may be positioned within the same room as the imaging device. Further, the syringe pump 10 may be positioned within the imaging device. For example, the syringe pump 10 may be configured to be positioned within a bore of an MRI scanner. The position of the device at, near, or within the imaging device eliminates the need for long tubes to connect the syringe 20 to the subject. The syringe pump 10 may be configured to deliver fluid from at least one syringe 20 at a desired rate without the need for programming. The syringe pump 10 may be configured to deliver a contrast agent from the syringe 20 at a substantially constant rate. In exemplary aspects, it is contemplated that a substantially constant rate may be a constant rate. In further exemplary aspects, it is contemplated that a substantially constant rate may deviate (upwardly or downwardly) from the desired rate during a portion of the contrast agent flow by up to 25 percent, up to 20 percent, up to 15 percent, up to 10 percent, up to 5 percent, or up to 1 percent.

In exemplary aspects, and with reference to FIGS. 1 and 1A, the syringe pump 10 may include at least one syringe holder 100. Each syringe holder 100 may be configured to hold a syringe 20. The syringe pump 10 may include a rack 40 configured to interact with and drive the plunger 22 of the syringe 20. The syringe pump 10 may include an engagement structure 50 configured to interact with the rack 40 to control the rate at which the plunger 22 is depressed into the barrel 24 of the syringe 20 thereby controlling the rate at which the syringe contents are injected. The syringe pump 10 may include components to activate the system. Optionally, these components may include a lock 110, a control cable 112, and a controller 120. The syringe pump 10 may include a rigid support. Optionally, the rigid support may be a base plate 130. Exemplary components of the syringe pump 10 and movement of such components within the system are further described herein.

The base plate 130 may provide a rigid support to the other components of the syringe pump 10. Each component may be connected or coupled (optionally, movably coupled) to the base plate 130. In an aspect, the syringe holder 100, the lock 110, and the engagement structure 50 may be connected to the base plate 130. The base plate 130 may include tracks to receive a gliding mechanism of the rack 40 to allow the rack to slide along the base plate 130. As shown in FIG. 1, the base plate 130 may also include a brace structure 132 that acts as a brace point (e.g., stop surface) for one end of a spring 30 while an opposing end of the spring 30 is free to move to allow the spring 30 to compress and expand along a first axis. The first axis may be an axis parallel or substantially parallel (within 10 degrees, within 5 degrees, or within 1 degree of parallel) to a length L of the syringe pump 10. Alternatively, with reference to FIGS. 1A-1B, the base plate 130 or a spring plate 129 connected to the base plate 130 may comprise at least one axle 133. Each axle 133 may be configured to connect to a spring 300. Each component of the syringe pump 10 may be completely metal free (completely free of metal). For example, each component may be made of, consist of, or consist essentially of plastics or other non-metal and/or non-magnetic materials. The syringe pump 10 may operate by itself to deliver a contrast agent or other fluid from at least one syringe 20 to a subject, such as a patient, or to an imaging phantom, inside an imaging device. The syringe pump 10 may be activated at a distance via the controller 120, which can optionally be in communication with the syringe pump 10 through a control cable 112. The syringe pump 10 may be powered by the spring 30. The injection rate from the syringe 20 may be controlled by the engagement structure 50.

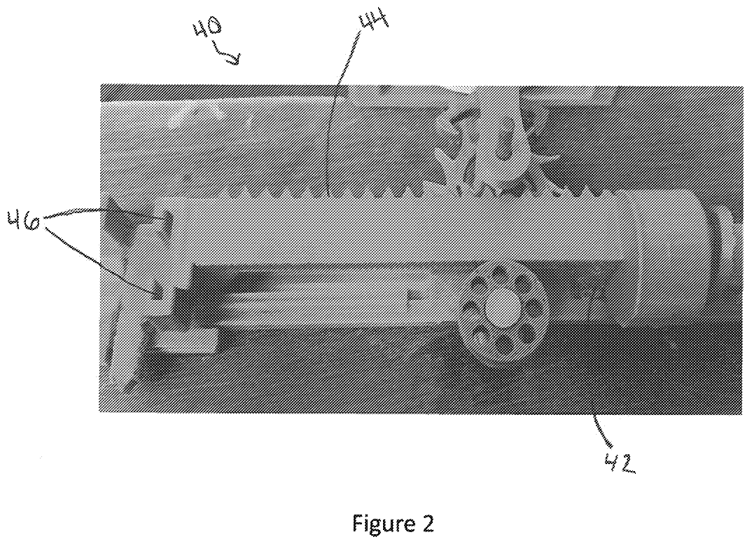

In exemplary aspects, and with reference to FIGS. 1 and 2, the rack 40 may be positioned between the spring 30 and the syringe holder 100. Alternatively, and with reference to FIGS. 1A and 1B, the rack 40 may be positioned elsewhere. In one aspect, the rack 40 may be positioned between the lock 110 and the syringe holder 100. The rack 40 may be configured to transmit power from the spring 30, 300 to a syringe plunger 22. The rack 40 may also be configured to move along the base plate 130 or a rack base 131. The rack 40 may include a mechanism to allow the rack 40 to glide along the base plate 130 or the rack base 131. For example, and with reference to FIGS. 2 and 1B, the rack 40 may include feet 42 that glide along tracks in the base plate 130 or the rack base 131. In another aspect not shown, the rack 40 may include bearings or wheels to move along a rail on the base plate 130 or rack base 131. The rack 40 may be configured to interact and engage with the engagement structure 50. The rack 40 may interact and engage with the engagement structure 50 via teeth 44 along a side or edge of the rack 40. The rack 40 may also include at least one slot 46 configured to hold a flange 26 of a plunger 22 of a syringe 20 positioned in the syringe holder 100. As the rack 40 moves the plunger 22 of the syringe 20 may move. The rack 40 may thereby drive the plunger 22 in and out of a stationary syringe barrel 24 held by the syringe holder 100.

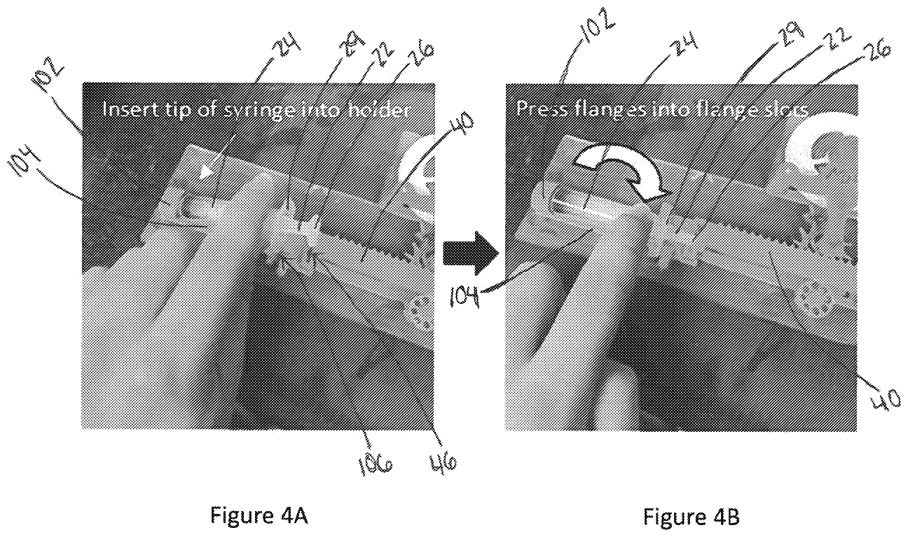

With reference to FIGS. 1, 1B, and 3-4B, a syringe holder 100 may be positioned adjacent to the rack 40. The syringe holder 100 may include a tip holder 102 configured to hold the tip 28 of the syringe 20, a barrel holder 104 configured to hold the barrel 24 of the syringe 20, and at least one slot 106 configured to hold a flange 29 of the barrel 24 of the syringe 20. The tip holder 102 may be comprised of an open archway. The barrel holder 104 may comprise a receptacle that defines a half-pipe section configured to cradle the barrel 24 of the syringe 20. The syringe holder 100 may be connected to the base plate 130 to remain stationary. The syringe holder 100 may hold the syringe 20 in place while the syringe pump 10 is in use. The syringe holder 100 may allow a syringe 20 to be quickly and easily installed prior to use and removed after use. For example, with reference to FIGS. 4A-4B, the tip 28 of the syringe 20 may be inserted into the opening of the tip holder 102. The flange 29 of the barrel 24 of the syringe 20 may then be pressed down into the slots 106 of the syringe holder 100 with the barrel 24 of the syringe 20 cradled by the barrel holder 104. At approximately the same time, the flange 26 of the plunger 22 of the syringe 20 may be pressed into the slots 46 of the rack 40. The syringe holder 100 may allow for easy installation and removal while providing a secure hold of the syringe 20 while the syringe pump 10 is in use. The syringe holder 100 may secure the syringe 20 in place while the rack 40 moves the plunger 22 in or out of the barrel 24 of the syringe 20.

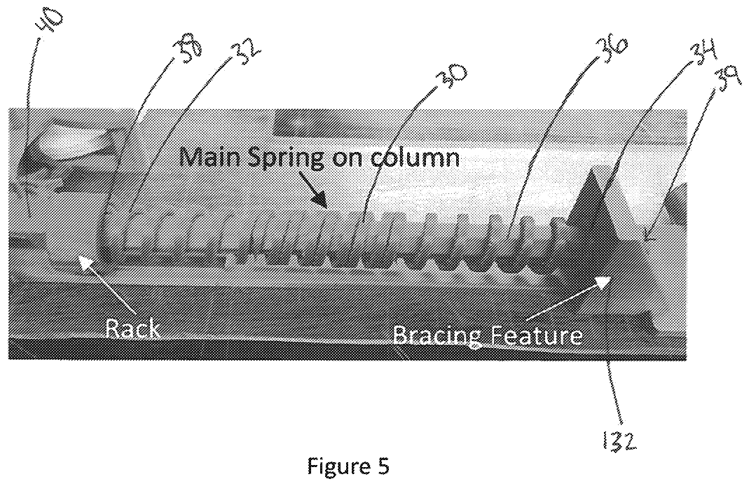

With reference to FIGS. 1, 1A, 1B and 5, a spring 30, 300 may power and/or energize the syringe pump 10. As shown in FIGS. 1A and 1B, more than one spring 30 may be used. In one example, two or more springs 30 may be connected and/or intertwined or otherwise provided in an overlapping configuration. The number of springs 30, 300 may depend on the elasticity of each spring 30, 300. In one aspect, a spring 30, 300 may be added after the elasticity of an original spring 30, 300 loses elasticity.

In one aspect, and with reference to FIGS. 1 and 5, the spring 30 may be a helical spring. In one aspect, as shown in FIGS. 1 and 5, the spring 30 may be positioned between the rack 40 and a brace structure 132 attached or coupled to the base plate 130. A first end 32 of the spring 30 may be free to move along the first axis. The second end 34 may be secured to the base plate 130. The second end may be fixed to the brace structure 132. The first end 32 of the spring 30 may move along the first axis to compress and extend the spring 30 along the first axis. As the first end 32 of the spring 30 moves between compressed and uncompressed positions, the rack 40 may move simultaneously between a first position, corresponding to when the spring 30 is compressed, and a second position, corresponding to when the spring 30 is uncompressed or extended. The spring 30 may further include a column 36 extending through the center or approximate center of the spring 30 along the first axis. The column 36 may be rigid. The column 36 may keep the spring 30 in position and prevent the column 36 from moving along an axis that is not the first axis. The column 36 may be configured to prevent the spring 30 from buckling when the spring 30 is compressed. The rack 40 may be connected or coupled to the spring 30 via a first end 38 of the column 36.

In one aspect, and with reference to FIGS. 1A and 1B, the spring 300 may be a spiral spring. A first end 302 of the spring 300 may be connected to the brace structure 132. In one aspect, the brace structure 132 may comprise a bearing 137 and axle 133. The spring 30 may be wound around the bearing 137 and axle 133. A second end 304 of the spring 30 may be connected to an outer ring 135. The outer ring 135 may rotate about the axle 133, wherein rotating the outer ring 135 in a first direction, for example counter-clockwise, wraps the spring 300 more tightly around the axle 133 compressing the spring 300, and rotating the outer ring 135 in a second direction, for example clockwise, loosens the spring 300 around the axle 133 expanding the spring 300. The outer ring 135 may comprise teeth 136 on the exterior surface. The teeth 136 of the outer ring 135 may be configured to mesh with (e.g., complementarily engage) teeth 342 of a secondary rack 340. The secondary rack 340 may be connected to the rack 40. The secondary rack 340 may be configured to move along the first axis with the rack 40. In one aspect, the secondary rack 340 may slide along the first axis via bearings 137. The bearings 137 may be configured to rotate about projections 341 connected to the base plate 130 or the spring plate 129. The secondary rack 340 and the rack 40 may move simultaneously or substantially simultaneously between a first position, corresponding to when the spring 30 is compressed or wound more tightly around the axle 133, and a second position, corresponding to when the spring 30 is uncompressed or more loosly wrapped around the axle 133. When the secondary rack 340 is moved from the second position to the first position, the teeth 342 of the secondary rack 340 engage the teeth 136 of the outer ring 135 of the spring 300 to rotate the outer ring 135 and compress the spring 300. When the spring 300 expands and rotates in the opposite direction, the teeth 136 of the outer ring 135 engage the teeth 342 of the secondary rack 340 to move the secondary rack 340 and the rack 40 connected to the secondary rack 40 from the first position to the second position.

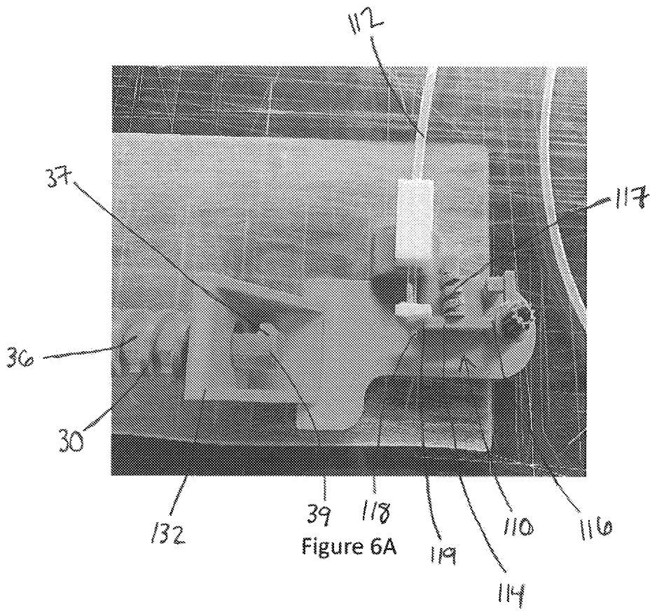

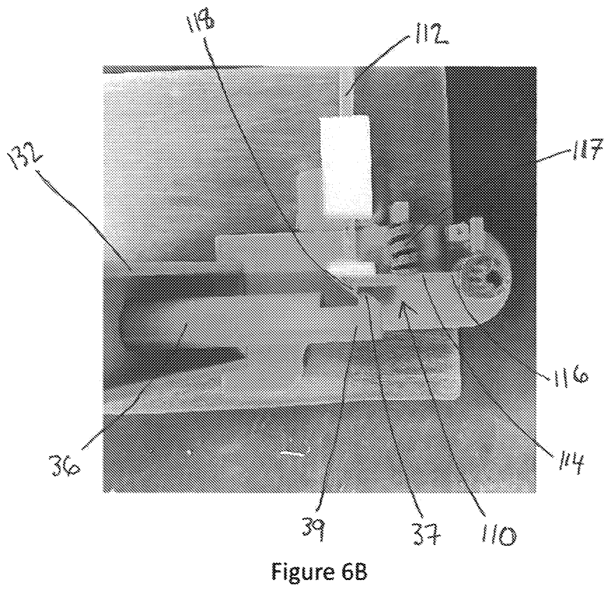

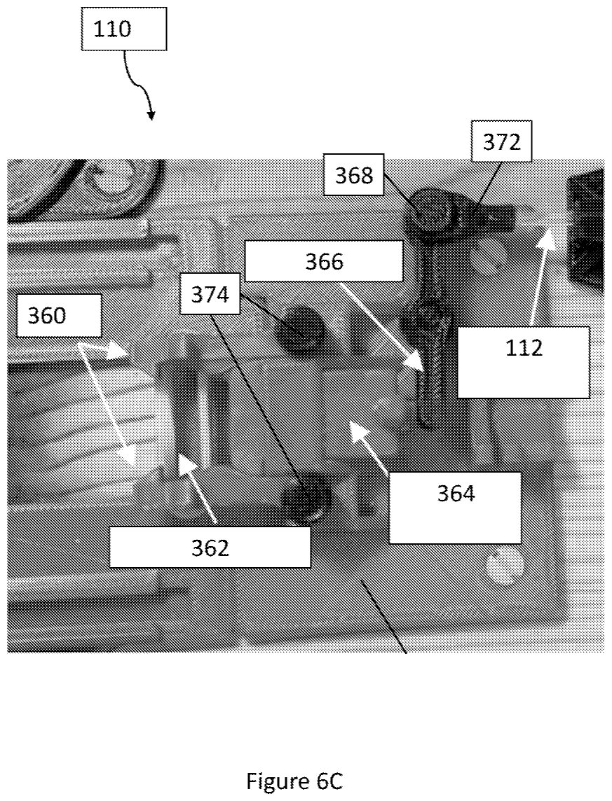

In one aspect, with reference to FIGS. 6A-6B, a lock 110 may engage with a second end 39 of the column 36. In one aspect, with reference to FIGS. 1A and 6C, the lock 110 may engage with the rack 40. More specifically, the lock 110 may engage with a projection 41 extending from the rack 40. The lock 110 may engage with the column 36 or the rack 40 to keep the spring 30, 300 compressed and hold the device in a loaded position. When the column 36 or rack 40 disengages with the lock 110, the spring 30, 300 may extend. As the spring 30, 300 extends, the energy may be converted to power the rack 40 and move the rack 40 from the first position to the second position along the first axis.

In one aspect, with reference to FIGS. 6A and 6B, the lock 110 may be a spring-lock. Further, the lock 110 may be a spring-loaded clip. The lock 110 may include a base plate 114. The base plate 114 may be able to rotate about a first end 116 of the base plate 114. The base plate 114 may include a latch 118. The latch 118 may be located on a surface of a second end 119 of the base plate 114. The lock 110 may include a spring 117 that puts pressure on the base plate 114 so that the latch 118 on the base plate 114 engages or clips with a latch 37 on the column 36. The second end 119 of the base plate 114 may also be attached to a control cable 112. When the control cable 112 is pulled, the tension is applied to the base plate 114 thereby compressing the spring 117 and rotating the base plate 114 away from the latch 37 on the column 36. When the base plate 114 rotates, the latch 37 on the base plate 114 may disengage or unclip from the latch 37 on the column 36. The column 36 may then move along the first axis away from the lock 110 as the spring 30 expands and pulls the column 36.

In one aspect, with reference to FIG. 6C, the lock 110 may comprise a pair of clips 360 configured to engage the projection 41 of the rack 40. The clips 360 may be configured to pivot about pins (e.g., caps) 374. In one aspect, a clip 360 may be positioned on either side of the projection 41 when the clips 360 are engaged with the projection 41. The lock 110 may comprise an elastic cable 362 configured to biase the pair of clips 360 towards one another. The lock 110 may further comprise a release pin 364 positioned between the pair of clips 360. The lock 110 may comprise a lever arm 366 configured to pivot about an axle 368 and pin 370 (shown in FIG. 1B) between a first position not engaging the release pin 364 and a second position engaging the release pin 364. The lever arm 366 may be attached to the control cable 112 via an elbow 372. The control cable 112 may be configured to pivot the lever arm 366 via the elbow 372 from the first position to the second position. When the control cable 112 is pulled, the tension pivots the lever arm 366 from the first position to the second position pushing the release pin 364 towards the pair of clips 360. As the release pin 364 moves towards the clips 360, the release pin 364 pushes the clips 360 away from each other thereby stretching the elastic cable 362. When the clips 360 move away from each other, the clips 360 move outwardly away from the projection 41 of the rack 40 thereby disengaging and releasing the projection 41 and rack 40. The rack 40 may then move along the first axis away from the lock 110 as the spring 300 expands and pulls the secondary rack 340 and the rack 40.

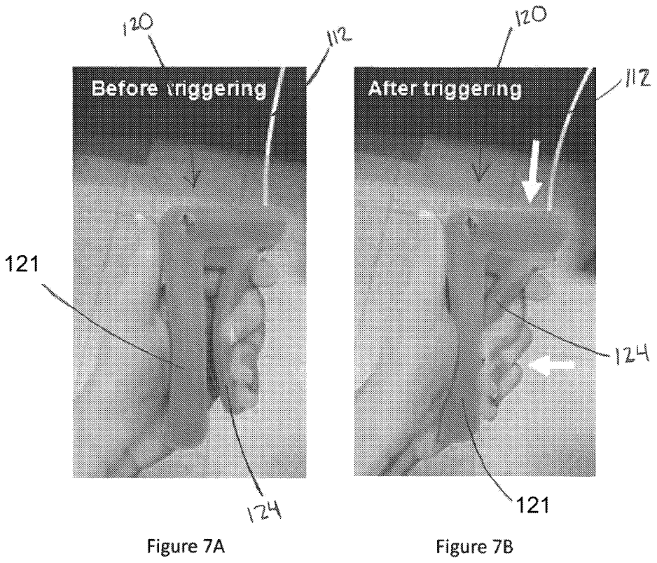

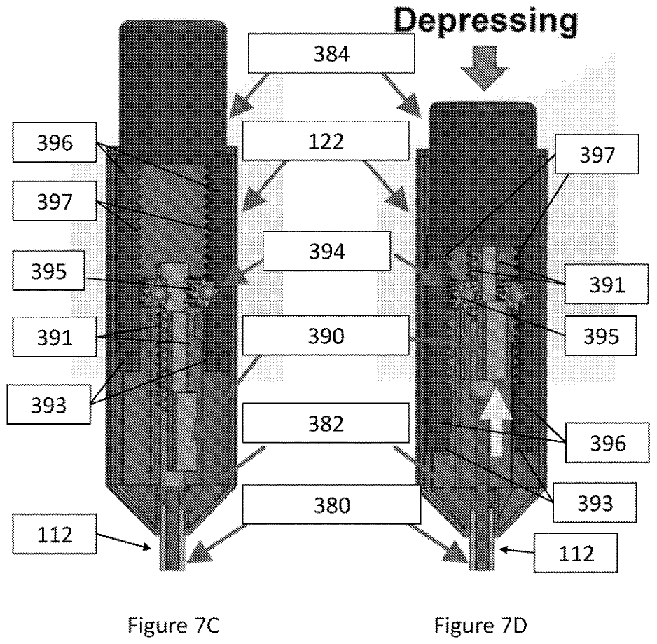

With reference to FIGS. 1, 1A, 7A, 7B, 7C, and 7D, the control cable 112 may run from the lock 110 to the controller 120. The control cable 112 may include an outer sleeve 380 and an inner cable 382. The cable-in-sleeve configuration may allow the lock 110 to be activated from a distance while allowing the control cable 112 to remain flexible and portable. Optionally, the inner cable may be a fiber optic cable, and the outer sleeve may be a polytetrafluoroethylene (PTFE) tube. The materials of the inner cable and the outer sleeve may have a low friction coefficient. The control cable 112 may be resistant to locking up or binding, even if a loop or kink is put in it, and may be durable enough to resist damaging compression (for example, in response to a downward and/or crushing force) due to the low friction coefficient between the materials and/or the stiffness of the inner cable. It is further contemplated that the inner cable (e.g., a fiber optic cable) can have sufficient stiffness to resist bending and kinking.

The controller 120 may be used to unlock and lock the lock 110. In one aspect, as shown in FIGS. 7A and 7B, the controller 120 may include a body 121, a handle 124, and a spring (not shown). Alternatively, as shown in FIGS. 7C and 7D, the controller 120 may include a body 122 and a push button 384. The outer sleeve of the control cable 112 may be attached to the body 121, 122 of the controller 120.

In one aspect, shown in FIGS. 7A and 7B, the inner cable of the control cable 112 may be attached to the handle 124 of the controller 120. The handle 124 of the controller 120 may be configured to be compressed. For example, the handle 124 may be configured to be squeezed by a hand of a user or operator. When compressed, tension may be applied to the inner cable of the control cable 112. The tension on the inner cable may pull the inner cable within the stationary outer sleeve of the control cable 112. When the inner cable moves, the inner cable may pull on the base plate 114 of the lock 110 which decouples the lock 110 from the spring column 36 and allows the spring 30 to expand. When the handle 124 is released and no longer compressed, the spring in the controller 120 and the spring 117 in the lock 110 may push the base plate 114 of the lock 110 back in its resting position. The lock 110 may latch with the second end 39 of the column 36 when the base plate 114 is in its resting position.

In one aspect, shown in FIGS. 7C and 7D, the inner cable 382 may be coupled to a first rack 390 positioned within the body 122. The first rack 390 may slide along grooves (not visible) within the interior surface of the body 122 to move toward and away from the push button 384. The first rack 390 may comprise at least one set of teeth 391. Each set of teeth 391 may be configured to mesh and engage with teeth 395 of a pinion 394. The teeth 391 of each pinion 394 may also be configured to mesh and engage with teeth 397 of a second rack 396 connected to the push button 384. When the push button 384 is depressed by a user, each second rack 396 may move further into the body 122 with the push button 384. When each second rack 396 moves further into the body 122, the teeth 397 of each second rack 396 may engage with and rotate the pinion 394 about the pinion's center axis. As the pinion 394 rotates, the teeth 395 of the pinion 394 engage with the teeth 391 of the first rack 390 to move the first rack 390 towards the push button 384. As the first rack 390 moves up towards the push button 384, the inner cable 382 is pulled, resulting in the lever arm 366 of the lock 110 pivoting to push the release pin 364 to unlock the rack 40 and activate the device. When the push button 384 is released, the push button 384 is biased outwardly away from the inside of the body 122. In one aspect, the ends 393 of the second rack 396 may be connected to elastic cables connected to the body 122 which bias the push button 384 outwardly away from the inside of the body 122.

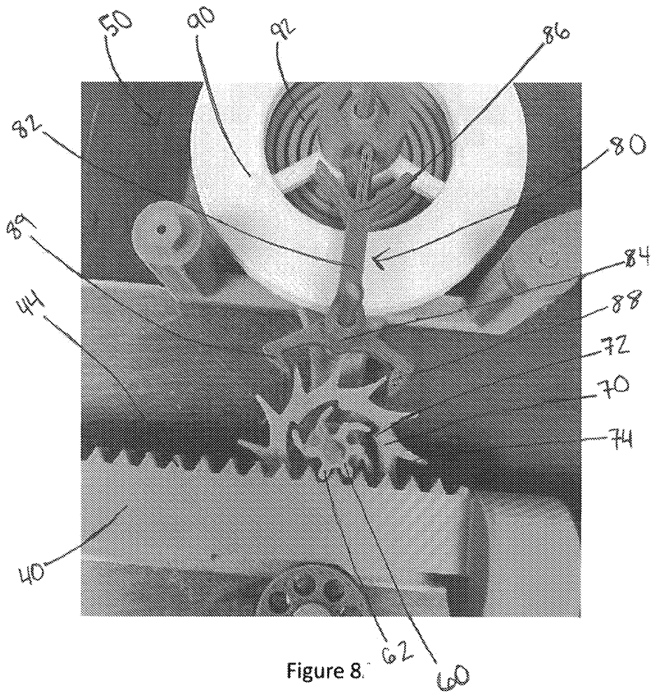

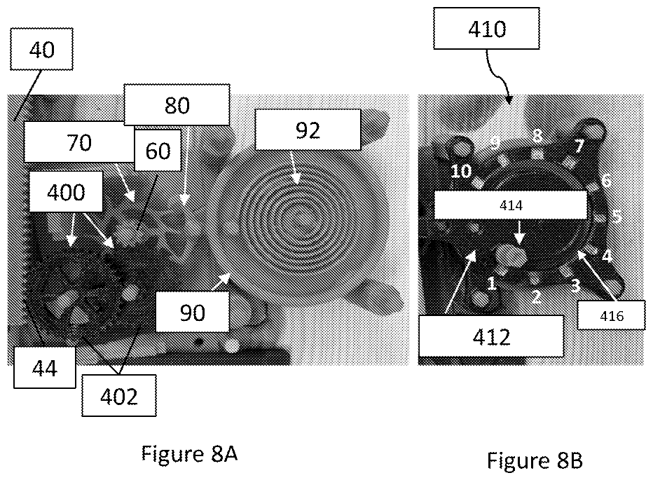

With reference to FIGS. 1, 1A, 1B, 8, and 8A, an engagement structure 50 may control the speed at which the rack 40 moves from the first position to the second position. In one aspect, the engagement structure 50 may comprise a gear arrangement. Optionally, the gear arrangement may comprise an escapement mechanism. As can be appreciated, during use of the disclosed syringe pump 10, a high accuracy of an infusion rate of the dispensing fluid, for example, a contrast agent, may be critical. Thus, it may be important that the syringe pump 10 has a reliable dispensing or injection rate. The engagement structure 50 may allow the linear force of the spring 30 to be controlled so that the injection rate may be specified and controlled rather than relying on the spring force alone. Optionally, the engagement structure 50 may comprise a ratcheting pinion 60, an escape wheel 70, a control arm 80, and a flywheel 90. However, it is contemplated that other mechanical structures, such as ratchet structures, can be employed.

In one aspect, with reference to FIGS. 1 and 8, the ratcheting pinion 60 of the engagement structure 50 may engage and interact with the rack 40. In one aspect, with reference to FIGS. 1A and 8A, the ratcheting pinion 60 may engage and interact with a gear set 400. The gear set 400 may include one or more gears. The ratcheting pinion 60 may be configured to rotate. The ratcheting pinion 60 may include teeth 62. The teeth 62 of the ratcheting pinion 60 may mesh with the teeth 44 of the rack 40. Alternatively, as shown in FIG. 8A, the teeth 62 of the ratcheting pinion 60 may mesh with teeth 402 of a gear of the gear set 400. Teeth 402 of a gear of a gear set 400 may mesh with the teeth 44 of the rack 40. The gear set 400 may be one or more reduction gears that connect the rack 40 to the escape wheel 70. Different sized gears may be selected based on the desired speed of the device and the desired injection rate. As the rack 40 moves from the first position to the second position, the teeth 44 of the rack 40 rotate the ratcheting pinion 60. In one aspect, the rack 40 rotates the gear set 400 to rotate the ratcheting pinion 60. In one aspect, as the rack 40 moves from the first position to the second position, the teeth 44 of the rack 40 may engage with the teeth 62 of the ratcheting pinion 60 to rotate the ratcheting pinion 60 in a first or forward (e.g., clockwise) direction. In one aspect, as the rack 40 moves from the first position to the second position, the teeth 44 of the rack 40 may engage with teeth 402 of the gear set 400 to rotate the racheting pinion 60 in the first or forward direction. The ratcheting pinion 60 transmits power from the spring 30, 300 to the escape wheel 70. The ratcheting pinion 60 may engage with the escape wheel 70 as it rotates. The teeth 62 of the ratcheting pinion 60 may engage with the escape wheel 70 as the ratcheting pinion 60 rotates forward (e.g., clockwise). The shape and design of the teeth 62 of the ratcheting pinion 60 may allow the teeth 62 of the ratcheting pinion 60 to slip past and not engage with the ratcheting pinion 60 if the ratcheting pinion 60 is rotated in a reverse or backward (e.g., counterclockwise) direction. This feature may allow a user or operator to push the rack 40 from the second position to the first position in order to compress the spring 30, 300 and load the syringe pump 10 for use without engaging the escape wheel 70.

The escape wheel 70 may receive power from the ratcheting pinion 60 and interface and engage with the control arm 80. The escape wheel 70 may be a gear. The escape wheel 70 may have a first set of teeth 72 on an interior surface of the escape wheel 70. The first set of teeth 72 may interact and mesh with the teeth 62 of the ratcheting pinion 60. As the ratcheting pinion 60 rotates, the ratcheting pinion 60 teeth 62 may push the first set of teeth 72 of the escape wheel 70 to rotate the escape wheel 70. For example, as the ratcheting pinion 60 rotates clockwise, the ratcheting pinion 60 may force the escape wheel 70 to rotate clockwise. The escape wheel 70 may have a second set of teeth 74 on an exterior surface of the escape wheel 70. The escape wheel 70 may interact with the control arm 80. In one aspect, the second set of teeth 74 may be long, curved teeth on the exterior surface to contact and interact with the control arm 80. When the escape wheel 70 rotates, the second set of teeth 74 of the escape wheel 70 may push the control arm 80. At least one tooth of the second set of teeth 74 of the escape wheel 70 may slide past the control arm 80. As the at least one tooth of the escape wheel 70 slips past the control arm 80, the control arm 80 may slide back into contact with another tooth of the escape wheel 70, thereby stopping the escape wheel 70 from rotating. When the escape wheel 70 stops rotating, the escape wheel 70 may stop the ratcheting pinion 60 from rotating. When the ratcheting pinion 60 stops rotating, the ratcheting pinion 60 may stop the rack 40 from moving from the first position to the second position. This process of starting and stopping the escape wheel 70 is what slows the motion from the spring 30, 300 down, and therefore the rack 40, to a controlled speed.

The control arm 80 may have at least two teeth which may be configured to engage and interact with the escape wheel 70. The control arm 80 may comprise a stem 82 having a first end 84 and a second end 86. The first end of the control arm 80 may include a first tooth 88 and a second tooth 89 as best shown in FIG. 8. The second end 86 of the stem 82 may be configured to engage and interact with the flywheel 90. The control arm 80 may be configured to rotate. When the escape wheel 70 pushes the first end 84 of the stem 82 of the control arm 80, the second end 86 of the stem 82 of the control arm 80 may rotate slightly and engage the flywheel 90.

The flywheel 90 may be a disk that is configured to rotate. In one aspect, the flywheel 90 may be a flat disc with sections near the center removed to increase its relative rotational inertia as best shown in FIG. 8. The flywheel 90 may comprise a spring 92. The spring 92 may be attached to the flywheel 90. In one aspect, the spring 92 may be a balance spring. Further, the spring 92 may be a spiral spring. A first end of the spring 92 may be attached to the flywheel 90 at the flywheel's center or approximate center. The second end of the spring 92 may be attached to the flywheel 90 at the flywheel's edge or approximate edge. The spring 92 may be configured to absorb and rebound the energy from the flywheel 90 as the flywheel 90 rotates.

The flywheel 90 may be configured to transfer the energy from the control arm 80 into the spring 92. When the control arm 80 interacts with the flywheel 90, the flywheel 90 may be forced to rotate. As the flywheel 90 rotates, the spring 92 may spool up or compress, absorbing and storing the energy. Once all the energy is absorbed into the spring 92 or the spring 92 is fully compressed or spooled, the spring 92 may rebound or expand. When the spring 92 rebounds or expands, the spring 92 may reverse the direction of rotation of the flywheel 90 and force the flywheel 90 to push back the control arm 80 in a second or opposite direction. When the control arm 80 moves in the second or opposite direction, the control arm 80 may stop the escape wheel 70 from rotating.

The weight and relative inertia of the flywheel 90 may work in conjunction with the spring 92 to control the speed at which the engagement structure 50 operates. Having a higher relative rotational inertia may allow more of the energy to be transferred, stored, and used again. The weight and relative inertia may be selected to produce a desired injection rate of the syringe pump 10. The heavier the flywheel 90 or the higher inertia of the flywheel 90 may result in slower cycles or injection rates. The lighter the flywheel 90 or the lower the inertia of the flywheel 90 may result in faster cycles or injection rates. The stiffness of the balance spring 92 may also impact the speed at which the engagement structure 50, and thereby the syringe pump 10, operates. A stiffer balance spring 92 may generally produce faster cycles.

In one aspect, shown in FIG. 8B, the engagement structure 50 may further comprise a speed controller 410. The speed controller 410 may be positioned over the flywheel 90 and balance spring 92. The speed controller 410 may comprise a slotted plate 412 and control pin 414. The slotted plate 412 may include a slot 416 configured to receive the control pin 414. The slot 416 may be arc shaped. In one aspect, the slot 416 may overlay at least a portion of the strip or wire that forms the exterior or outermost coil of the spring 92. The pin 414 may be inserted at different positions along the slot 416. When inserted, the pin 414 may be configured to secure the strip or wire at a specific location resulting in a descrease in the spring's 92 effective length. The effective length of the coiled strip or wire of the spring 92 is inversely proportional to the spring 92 oscillation frequency, and consequently, the injection speed. The control pin 414 may shorten the spring's effective length, increasing its effective stiffness, which causes the escapement mechanism of the gear arrangement to run more quickly. In one aspect, the slotted plate 412 may include indicators to indicate positions and associated speeds along the spring 92. For example, as shown in FIG. 8B, the slotted plate 412 may include indicators (e.g., numbers indicating an angular orientation, such as, for example and without limitations, indicators positioned every 30 degrees from and labeled from 1 to 10). For example, as shown in FIG. 8B, the injection speed is highest when the pin 414 is positioned at indicator 1 and lowest when the pin 414 is positioned at indicator 10.

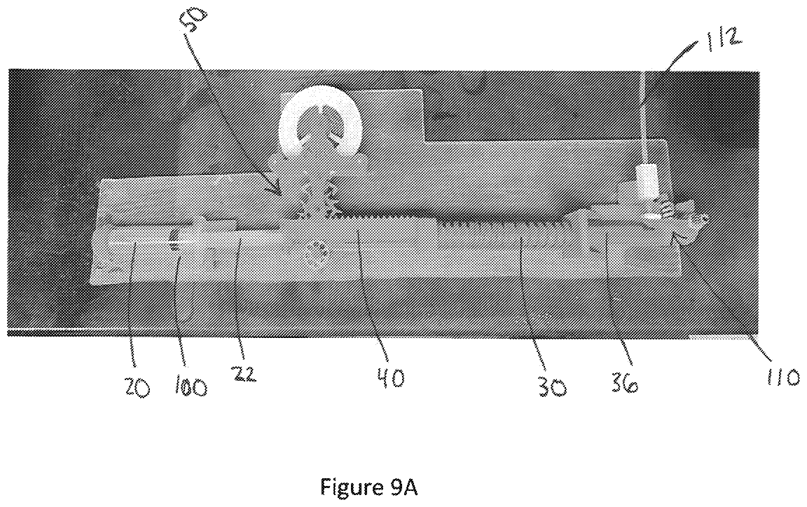

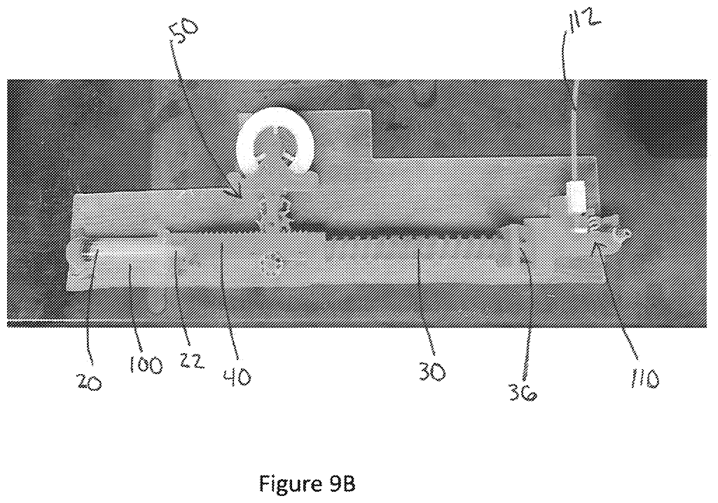

To begin operation of the syringe pump 10, the rack 40 may be slid or pushed back to compress the spring 30, 300 until the spring column 36 or rack 40 latches with the lock 110. This position prior to injection may be defined as the loaded position. With reference to FIG. 9A, a syringe 20 filled with fluid, such as a contrast agent, may be placed into the syringe holder 100 when the syringe pump 10 is in the loaded position. Tubes may be connected from the syringe 20 to a subject that may receive the contents of the syringe 20. An operator or user may activate the syringe pump 10 by squeezing the handle 124 or pushing the push button 384 of the controller 120. The controller 120 may pull the control cable 112 to pull back and disengage or unlock the lock 110. When the lock 110 is unlocked, the spring 30, 300 may expand and move the rack 40. The spring 30, 300 may move the rack 40 across the base plate 130 from the first position to the second position. As the rack 40 moves, the rack 40 drives the plunger 22 into the barrel 24 of the syringe 20. Further, while the rack 40 moves from the first position to the second position, teeth 44 on the rack 40 may mesh with the ratcheting pinion 60 or the gear set 400 of the engagement structure 50 to control how fast the rack 40 moves, and therefore, how fast the plunger 22 is driven into the barrel 24 of the syringe 20. With reference to FIG. 9B, the end of the operation of the syringe pump 10 may result with an uncompressed spring 30, 300, the rack 40 in the second position, and the plunger 22 depressed into the syringe 20 barrel 24. This operation may allow the syringe pump 10 to independently control the injection rate of the syringe contents to a subject.

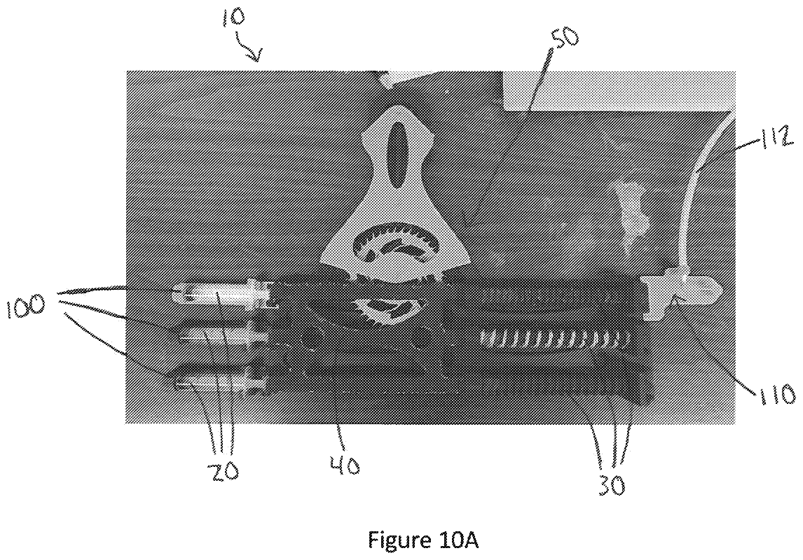

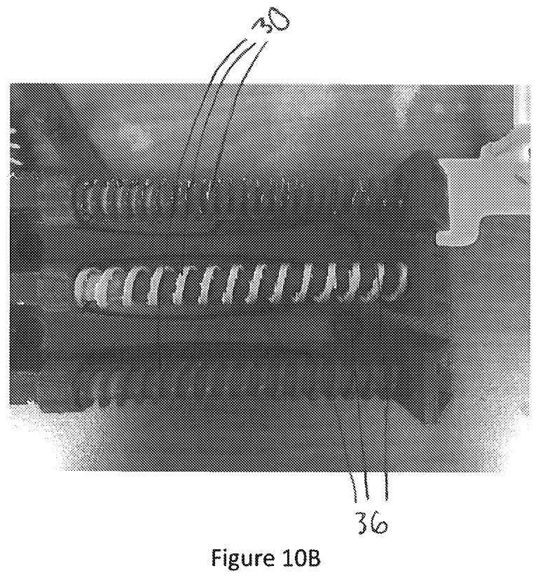

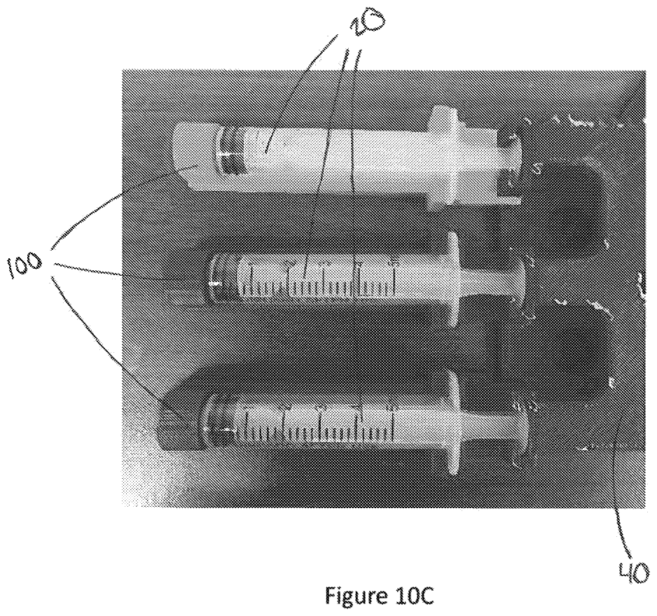

In one aspect, the syringe pump 10 may be modified to accommodate multiple syringes 20 that are each configured to provide a respective injection. This aspect may allow the contents of more than one syringe 20 to be delivered to multiple subjects. With reference to FIGS. 1A, 10A-10C, the syringe holder 100 and spring 30, 300 systems may be multiplied to accommodate additional syringes 20. The columns 36 or rack 40 of the spring 30, 300 systems may be connected or coupled together to allow the multiple springs 30, 300 to expand and contract at substantially the same time and rate, thereby leading to a substantially equal displacement of the plungers 22 of each of the respective syringes 20. The rack 40 may be enlarged to accommodate multiple syringes 20 and spring 30, 300 systems. The springs 30, 300 may be activated in unison to apply force to the rack 40 to dispense contents from the multiple syringes 20 at substantially the same infusion rate at the same time. This aspect may allow multiple simultaneous or substantially simultaneous injections.

Although the foregoing invention has been described in some detail by way of illustration and example for purposes of clarity of understanding, certain changes and modifications may be practiced within the scope of the appended claims.

1. A syringe pump comprising:
at least one spring configured to move between a compressed position and an uncompressed position;
a rack connected to the at least one spring and configured to move between a first position and a second position via the at least one spring, wherein the rack is in the first position when the spring is in the compressed position and in the second position when the spring is in the uncompressed position;
an engagement structure movably coupled to the rack and configured to control a speed at which the rack moves between the first position and the second position; and
a syringe holder configured to receive a syringe;
wherein the rack is further configured to engage with a plunger of the syringe and provide force to the plunger as the rack moves from the first position to the second position to drive the plunger into the syringe.

2. (canceled)

3. The syringe pump of claim 1, wherein the syringe pump consists of non-metallic material.

4. (canceled)

5. The syringe pump of claim 1, wherein the syringe pump is configured to be placed inside an Magnetic Resonance Imaging (MRI) bore.

6. The syringe pump of claim 1 further comprising a lock configured to hold the spring in the compressed position when locked and configured to release the spring when unlocked.

7. (canceled)

8. (canceled)

9. The syringe pump of claim 1 further comprising at least one second syringe holder configured to receive a second syringe, wherein the rack is further configured to engage with a second plunger of the second syringe and provide force to the second plunger as the rack moves from the first position to the second position to drive the second plunger into the second syringe.

10. The syringe pump of claim 1, wherein the engagement structure comprises:
a ratcheting pinion configured to interact with the rack, wherein the ratcheting pinion rotates as the rack moves from the first position to the second position;
an escape wheel configured to interact with the ratcheting pinion, wherein the escape wheel rotates as the ratcheting pinion rotates;
a control arm comprising a stem, wherein the escape wheel moves a first end of the stem in a first direction when the escape wheel rotates; and
a flywheel comprising a balance spring, wherein a second end of the stem rotates the flywheel and spools the balance spring when the first end of the stem moves in the first direction, wherein the spooled balance spring is configured to rebound and move the first end of the stem in a second direction, wherein the first end stops the escape wheel and ratcheting pinion from rotating and the rack from moving from the first position to the second position when moved in the second direction.

11.-21. (canceled)

22. An imaging system comprising:
an imaging device having a bore configured to receive at least a portion of at least one subject; and
a syringe pump configured for positioning within the bore of the imaging device and configured to administer a fluid during an imaging procedure, wherein the syringe pump consists of non-metallic material.

23. (canceled)

24. (canceled)

25. The imaging system of claim 22, wherein the imaging device is an MRI scanner.

26. The imaging system of claim 22, wherein the syringe pump comprises:
a spring configured to move between a compressed position and an uncompressed position;
a rack connected to the spring and configured to move between a first position and a second position via the spring, wherein the rack is in the first position when the spring is in the compressed position and the second position when the spring is in the uncompressed position;
an engagement structure movably coupled to the rack and configured to control a speed at which the rack moves between the first position and the second position; and
a syringe holder configured to receive a syringe;
wherein the rack is further configured to engage with a plunger of the syringe and provide force to the plunger as the rack moves from the first position to the second position to drive the plunger into the syringe.

27. The imaging system of claim 26, wherein the syringe pump further comprises a lock configured to hold the spring in the compressed position when locked and configured to release the spring when unlocked.

28. (canceled)

29. (canceled)

30. The imaging system of claim 26, wherein the syringe pump further comprises at least one second syringe holder configured to receive a second syringe, wherein the rack is further configured to engage with a second plunger of the second syringe and provide force to the second plunger as the rack moves from the first position to the second position to drive the second plunger into the second syringe.

31. The imaging system of claim 26, wherein the engagement structure comprises:
a ratcheting pinion configured to interact with the rack, wherein the ratcheting pinion rotates as the rack moves from the first position to the second position;
an escape wheel configured to interact with the ratcheting pinion, wherein the escape wheel rotates as the ratcheting pinion rotates;
a control arm comprising a stem, wherein the escape wheel moves a first end of the stem in a first direction when the escape wheel rotates; and
a flywheel comprising a balance spring, wherein a second end of the stem rotates the flywheel and spools the balance spring when the first end of the stem moves in the first direction, wherein the spooled balance spring is configured to rebound and move the first end of the stem in a second direction, wherein the first end stops the escape wheel and ratcheting pinion from rotating and the rack from moving from the first position to the second position when moved in the second direction.

32. (canceled)

33. A method comprising:
operating an imaging device; and
using a syringe pump in an imaging zone while the imaging device is operating, wherein the syringe pump consists of non-metallic material.

34. The method of claim 33, wherein the imaging zone is defined as a room within which the imaging device is located.

35. (canceled)

36. (canceled)

37. The method of claim 33, wherein the imaging device is an MRI scanner.

38. The method of claim 37, further comprising positioning the syringe pump in a bore of the MRI scanner.

39.-45. (canceled)

46. The syringe pump of claim 10, wherein the ratcheting pinion is configured to interact with the rack via a gear set.

47. The syringe pump of claim 10, wherein the engagement structure further comprises a speed controller, the speed controller comprising a slotted plate comprising a slot and a control pin configured to insert into the slot and secure the balance spring at a desired location along a length of the balance spring.

48. (canceled)

49. (canceled)

50. The syringe pump of claim 49, wherein the at least one spring comprises a spiral spring, the syringe pump further comprising a secondary rack connected to the rack, wherein the spiral spring comprises a toothed outer ring configured to rotate, the toothed outer ring engaging the secondary rack, wherein the second rack moves as the rack moves between the first position and the second position, wherein movement of the second rack causes the toothed outer ring to rotate to compress the spring, and wherein rotation of the toothed outer ring as the spring expands causes the second rack to move.
